(19) United States
(12) Patent Application Publication
(10) Pub. No.: US 2023/0195716 A1
(43) Pub. Date: Jun. 22, 2023

(54) AUTOMATED RECONCILIATION OF CENSUS DATA

(71) Applicant: MatrixCare, Inc., Bloomington, MN (US)

(72) Inventors: Joseph CAUDILL, Bloomington, MN (US); Danny WONG, Bloomington, MN (US); Bill KILBURN, Bloomington, MN (US); Adam MATHEWS, Bloomington, MN (US); Annie MCDANIEL, Bloomington, MN (US); Anwar KEMAL, Bloomington, MN (US)

(21) Appl. No.: 18/086,337

(22) Filed: Dec. 21, 2022

Related U.S. Application Data
(60) Provisional application No. 63/292,955, filed on Dec. 22, 2021.

(51) Int. Cl. G06F 16/23; G16H 10/60

(52) U.S. Cl. CPC G06F 16/2365; G16H 10/60

(57) ABSTRACT

Embodiments herein describe an automated reconciliation system that can detect census data discrepancies between two computing systems and perform automatic reconciliation. A first computing system is a source of truth for the census data, while a second computing system maintains its own copy of the census data. When its census data is updated, the first computing system can push census event notifications to the second computing system so it can update its copy of the census data. In one embodiment, the second computing system includes an error detector to identify when a census event received from the first computing system results in an unexpected census state. In response, the second computing system can perform an automatic reconciliation to correct the discrepancy by requesting a history of census events from the first computing system that can be used to rebuild the census data at the second computing system.

### BACKGROUND

### Field

Aspects of the present disclosure relate to automatically reconciling census data between two separate computing systems.

### Description of the Related Art

Many patient care facilities (e.g., hospitals, clinics, rehabilitation centers, long term care centers, etc.) generate census data to monitor the location of patients within the facility, or locations in different facilities of the same healthcare provider. The healthcare provider can have an admission system that maintains the census data. This census data may also be pushed to other computing systems so they can make intelligent decisions using the census data, such as marketing systems, customer satisfaction systems, medical insurance systems, pharmacy networks, and the like. These external or separate systems may maintain their own copies of the census data.

However, the census data maintained by the admission system may deviate overtime from the census data maintained by the external systems. For example, a network error may prevent an external system from receiving an update, or the external system may have been down for maintenance when the admission system sent a census update. When discrepancies are detected, they typically require a system administrator to fix. Manually reconciling the census data can require a substantial amount of time and is prone to human errors.

### SUMMARY

Certain embodiments provide a method that includes receiving, from a source of truth via a network, a census event for updating census data stored at a computing system where the census data defining a census state for a person, determining that the census event causes the person to have an unexpected census state, transmitting, via the network, a reconciliation request to the source of truth, receiving, via the network, a first history of census events from the source of truth corresponding to the person, deleting a second history of census events stored at the computing system for the person, and processing the first history of census events to rebuild the census data for the person stored at the computing system.

Certain embodiments provide a non-transitory computer readable medium comprising instructions to be executed in a processor, the instructions when executed in the processor perform an operation. The operation includes receiving, from a source of truth via a network, a census event for updating census data stored at a computing system where the census data defining a census state for a person, determining that the census event causes the person to have an unexpected census state, transmitting, via the network, a reconciliation request to the source of truth, receiving, via the network, a first history of census events from the source of truth corresponding to the person, deleting a second history of census events stored at the computing system for the person, and processing the first history of census events to rebuild the census data for the person stored at the computing system.

Certain embodiments provide a system that includes a processor and memory storing code which, when executed by the processor, performs an operation. The operation includes receiving, from a source of truth via a network, a census event for updating census data stored at a computing system where the census data defining a census state for a person, determining that the census event causes the person to have an unexpected census state, transmitting, via the network, a reconciliation request to the source of truth, receiving, via the network, a first history of census events from the source of truth corresponding to the person, deleting a second history of census events stored at the computing system for the person, and processing the first history of census events to rebuild the census data for the person stored at the computing system.

The following description and the related drawings set forth in detail certain illustrative features of one or more embodiments.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates a block diagram of an automatic reconciliation system, according to one embodiment.

FIG. 2 illustrates a block diagram of a computing system with a reconciliation engine, according to one embodiment.

FIG. 3 is a flowchart for performing automatic reconciliation of census data between two separate computing systems, according to one embodiment.

FIG. 4 illustrates a block diagram of an automatic reconciliation system, according to one embodiment.

FIG. 5 is a flowchart for periodically comparing census events in two copies of census data to identify discrepancies, according to one embodiment.

FIG. 6 illustrates a computing system, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Embodiments herein describe an automated reconciliation system that can detect census data discrepancies between two computing systems and perform automatic reconciliation, without human intervention. Not only does this save time (i.e., improves efficiency), it also improves the functioning of the computing systems by improving the accuracy of the census data so the computing systems can make more intelligent decisions when using the census data. That is, the automated reconciliation system can avoid human errors.

In one embodiment, a first computing system (e.g., a server, software application, data center, or cloud computing environment) is a source of truth for the census data. For example, the first computing system may be the admission system for a hospital that monitors the locations of admitted patients in the hospital. The second computing system may be, e.g., a marketing application that relies on having accurate census data in order to make decisions or recommendation. If, for whatever reason, there is a discrepancy between the census data stored at the first computing system and the census data stored at the second computing system, this can negatively impact the ability of the second computing system to make optimal decisions and recommendations.

When its census data is updated, the first computing system can push census event notifications to the second computing system so it can update its copy of the census data. In one embodiment, the second computing system includes an error detector to identify when a census event received from the first computing system results in an unexpected census state. For example, the census event may indicate that Patient A has been discharged from the hospital, but according to the census data stored in second computing system, Patient A has not yet been admitted into the hospital. Thus, the census state received from the first computing system is “unexpected” according to the census data already stored at the second computing system. Detecting an unexpected census state indicates there is a discrepancy between the census data stored at the two systems.

In response, the second computing system can perform an automatic reconciliation to correct the discrepancy. In one embodiment, the second computing system transmits a reconciliation request to the first computing system that lists the specific person (e.g., patient) where a discrepancy was detected. In response, the first computing system transmits the history of that person, which can include the full list of census events, to the second computing system. Using the history, the second computing system deletes the census data it has for the person and processes the received list of census events in order to rebuild the census data. In this manner, the second computing system can detect discrepancies in the census data and perform a reconciliation process to rebuild and replace its census data with census data from the source of truth—i.e., the first computing system. This automatic reconciliation process can improve the functioning of the second computing system by reducing the amount of time it takes to recover from discrepancies in census data and by improving the accuracy of the reconciliation process, relative to relying on a human. As a result, the second computing system can make better (e.g., more optimal) decisions based on the census data.

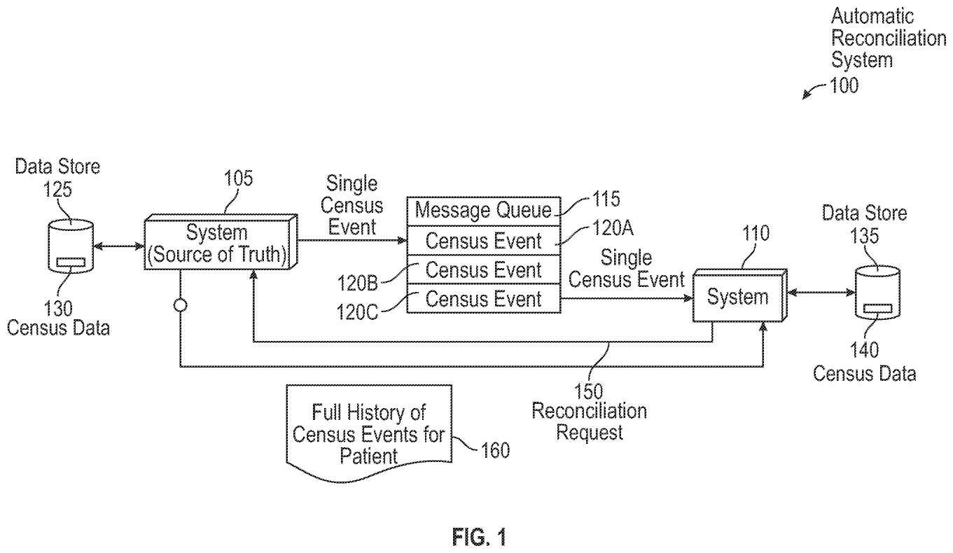

FIG. 1 illustrates a block diagram of an automatic reconciliation system 100, according to one embodiment. The system 100 includes a first computing system 105 that transmits census events to a second computing system 110. The computing systems 105, 110 can be different software applications, different software platforms, different servers, different cloud computing environments, and the like. For example, the first computing system 105 can be an admission system for a healthcare facility that monitors the location of the admitted patients. As the patients move to different locations within the facility (e.g., from the emergency room, to admissions, to x-ray, to the intensive care unit, to the burn unit, etc.), the admission system can update census data 130 stored in a data store 125 (e.g., a server, database, cloud storage system, etc.). For example, healthcare professionals may update the admission system each time the patient is moved to a different location. Or the admission system may be updated automatically by monitoring a location-aware band worn by the patient or some other location monitoring device.

In one embodiment, the census data 130 can include an electronic health record (EHR) for each patient in the facility. The EHR can list the census events for each patient. For example, an EHR for Patient A may indicate this patient was first admitted into the emergency room, then moved to an operation room, then moved to a recovery unit. Each of the three movements in the facility can be stored in the EHR as census events. The most recent census event in the EHR is the census state of the patient—i.e., their current location or status. For example, the census status for Patient A in this example is the recovery unit. If a patient has been discharged from the facility, their census status can be “discharged”, or “no longer at the facility”.

In this example, the census data 130 maintained by the computing system 105 serves as the source of truth. That is, other systems (e.g., the system 110) treat the census data 130 as a ground truth of the census data. In one embodiment, the goal of these systems is to ensure their census data matches the census data 130. As shown in FIG. 1, the computing system 105 transmits census events to the computing system 110 using a message queue 115. That is, each time the computing system 105 updates its census data for a patient (e.g., each time a patient moves to a different location in the facility, thereby triggering a census event), the computing system 105 transmits a census event 120 to the message queue 115.

The message queue 115 stores the census events 120 and then forwards these events 120 to the second computing system 110. While the system 105 could transmit the census events 120 directly to the computing system 110, using the message queue 115 helps to ensure that the computing system 110 is not overwhelmed by census updates. For example, the message queue 115 may forward the census events 120 at a constant rate to the second computing system 110 even though it receives the census events 120 in bursts from the first computing system 105. Further, while FIG. 1 illustrates forwarding the census events 120 only to the second computing system 110, there may be multiple other computing systems that want to receive the events 120. The message queue 115 can handle the task of distributing the events 120 to multiple destinations while the first computing system 105 needs to send the events only to the message queue 115. This may reduce the load on the first computing system 105.

In one embodiment, the message queue 115 is a first-in first out (FIFO) buffer that forwards the census events 120 to the second computing system 110 (and any other computing system) in the same order it receives them. However, the message queue 115 is not limited to any particular type of forwarding scheme.

In one embodiment, transmitting census events 120 reduces the amount of bandwidth used to ensure census data 140 for the second computing system 110 is updated to match or mirror the census data 130 for the first computing system 105, relative to transmitting the entire EHR for the patient each time their census state changes (e.g., each time the patient changes locations). The EHR can be a large file relative to the census events 120. Thus, transmitting the census events 120 each time census data (e.g., a patient's EHR) is updated is a relatively small data transaction relative to transmitting all the census data for a patient to the second computing system 110. This can reduce strain on the network (e.g., a local network or a public network) communicatively coupling the first and second computing systems 105, 110. Moreover, transmitting the census events 120 can improve the operation of the underlying hardware in the computing system 105. For example, transmitting the census events 120 can generate less strain on a network interface card and rely on smaller data queries to the data store 125 (e.g., memory) relative to transmitting all the census events for a patient each time their status is updated (e.g., transmitting the patient's EHR).

However, transmitting only the new census events 120 in response to a change to the census data 130 can result in the census data 140 in the data store 135 for the second computing system 110 becoming out of synch with the census data 130 for the first computing system 105. For example, the system 100 may have experienced a network failure that prevented the message queue 115 from receiving or forwarding one of the census events 120. Or the second computing system 110 may have been unavailable during a time when the message queue 115 forwarded it a census event 120. In any case, the census state for a patient in the census data 140 may not match the census state for that same patient in the census data 130.

Discussed in more detail below, when detecting a discrepancy in the census data 140 relative to the census data 130, the second computing system 110 performs a reconciliation process in order to correct the discrepancy in the census data 140. As shown, the second computing system 110 transmits a reconciliation request 150 to the first computing system 105, indicating the patient who has a census state in the census data 140 that does not match the census state of the patient in the census data 130. In response, the first computing system 105 pulls a full history 160 of the patient (e.g., the patient's EHR) from its census data 130 and forwards this history 160 to the second computing system 110 along a path in the network that bypasses the message queue 115. In one embodiment, the history 160 includes a full list of the census events for the patient (or resident).

The second computing system 110 can use the history 160 to correct the discrepancy in the census data 140. For example, the second computing system 110 can replace the census events stored for the patient in the census data 140 with the census events in the history 160. In this manner, the second computing system 110 ensures its census data 140 now matches the census data 130 of the first computing system 105.

In sum, the automatic reconciliation system 100 advantageously uses the relatively lightweight census events 120 to update the census data 140 to match the census data 130. However, when a discrepancy is detected, the system 100 can then automatically perform reconciliation where the relative heavyweight, full history 160 is transmitted between the two computing system 105, 110 in order to correct the discrepancy. In this manner, the strain on the network and the computing system 105, 110 is reduced relative to a system that transmits the full history of a patient each time their census state changes. Moreover, the system 100 can perform reconciliation without any human intervention which means discrepancies can be corrected almost as soon as they are detected and the second computing system 110 receives the full history 160 from the first computing system 105.

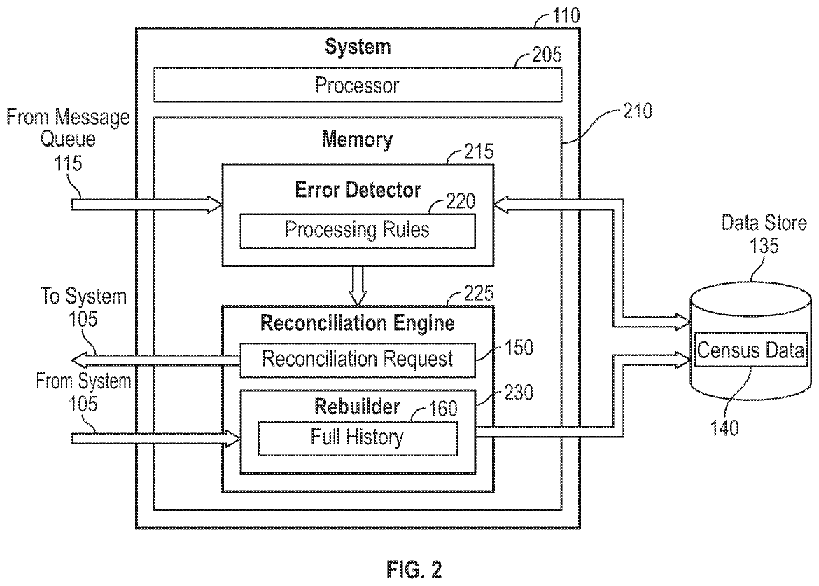

FIG. 2 illustrates a block diagram of the second computing system 110, according to one embodiment. As shown, the second computing system 110 includes a processor 205 and memory 210. The processor 205 (or processors) can be any electronic circuitry, including, but not limited to one or a combination of microprocessors, microcontrollers, application specific integrated circuits (ASIC), application specific instruction set processor (ASIP), and/or state machines, that communicatively couples to memory and controls the operation of the components in the second computing system 110 which can be software applications containing software (or firmware) code executed by the processor 205. The processor 205 may be 8-bit, 16-bit, 32-bit, 64-bit or of any other suitable architecture. The processor 205 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor 205 may include other hardware that operates software to control and process information. The processor 205 can execute software stored on the memory 210 to perform any of the functions described herein. The processor 205 controls the operation and administration of the second computing system 110 by processing information. The processor 205 is not limited to a single processing device and may encompass multiple processing devices.

The memory 210 in the second computing system 110 may store, either permanently or temporarily, data, operational software, or other information for the processors. The memory 210 may include any one or a combination of volatile or non-volatile local or remote devices suitable for storing information. For example, the memory 210 may include random access memory (RAM), read only memory (ROM), magnetic storage devices, optical storage devices, or any other suitable information storage device or a combination of these devices. The software stored in the memory 210 can include suitable set of instructions, logic, or code embodied in a computer-readable storage medium. For example, the software may be embodied in the memory, a disk, a CD, or a flash drive. In particular embodiments, the software may include an application executable by the processor 205 to perform one or more of the functions described herein.

In this example, the memory 210 includes an error detector 215 (e.g., a software application or module) and a reconciliation engine 225 (e.g., a software application or module). The error detector 215 identifies discrepancies between the census data 140 for the second computing system 110 and the census data for the first computing system (not shown). In one embodiment, the error detector 215 determines when a new census event received from the message queue 115 puts a patient or resident in an unexpected census state. For example, the census event may indicate that Patient A has been discharged from the hospital, but according to the census data 140 maintained by the second computing system 110, Patient A has not yet been admitted into the hospital. Or, the census event may indicate that Patient A is in an operation recovery unit but according to the census data 140 the Patient A has not yet been in an operation room. These examples illustrate how a new census event may place the patient in an unexpected census state.

The error detector 215 stores processing rules 220 to determine when the newly received census event triggers an unexpected census state. For example, the processing rules 220 can set certain prerequisite census events that must occur in a patient's EHR before the new census event. Using the examples above, a processing rule 220 may state that in order to process a new census event indicating the patient was discharged from a facility, the patient's EHR should already include a census event indicating the patient was admitted into the facility. The error detector 215 can retrieve the patient's EHR from the census data 140, determine whether there is census event indicating the patient was admitted, and if not, determine the new census event causes the patient to have an unexpected census state.

Similarly, a processing rule 220 may state that in order to process a census event indicating the patient was moved to a recovery unit, the patient's EHR should already include a census event indicating the patient was in an operation room. If not, the error detector 215 determines the new census event causes the patient to have an unexpected census state. This indicates there is a discrepancy between the census data 140 and the census data at the source of truth—e.g., the first computing system.

Census events are generally predictable and linear in time. Thus, the processing rules 220 can indicate what census events are prerequisites for other census events. While the examples above describe processing rules 220 that list a single prerequisite census event, a processing rule 220 may list multiple census events that should occur before the currently received census event. For example, before processing a census event indicating a patient is in the recovery room, the processing rule 220 for that event may indicate the patient's EHR should include census events indicating the patient was admitted into the facility and was in a pre-operation unit. If not, the error detector 215 determines the new census event would put the patient in an unexpected census state, and thus, there is a discrepancy.

If the error detector 215 identifies a discrepancy, the error detector 215 informs the reconciliation engine 225 of the name of the patient in the unexpected census state. The reconciliation engine 225 generates the reconciliation request 150 that is then forwarded to the first computing system 105. The reconciliation request 150 can include the name of the patient as well as other information.

In response to receiving the reconciliation request 150, the system 105 transmits the full history 160 of the patient to the second computing system 110. In one embodiment, the full history 160 may be the patient's EHR, but this is not a requirement. In another embodiment, the history 160 is the complete list of census events stored in the census data for the first computing system 105, which may not include other information that is typically stored in the EHR.

The reconciliation engine 225 includes a rebuilder 230 (e.g., a software application or module) that uses the history 160 to rebuild the patient's information in the census data 140. That is, the rebuilder 230 can use the history 160 to store all the census events for the patient in the census data 140. The census events in the history 160 can include the census event that caused the error detector 215 to detect the discrepancy and trigger the automatic reconciliation. Thus, once the census events provided by the history 160 are stored in the census data 140, the census data for that patient is now in the same state as the census data in the first computing system 105. Stated differently, the patient has the same census state in both computing systems, thereby completing the reconciliation process. The details of this reconciliation process are discussed in detail in the following flowchart.

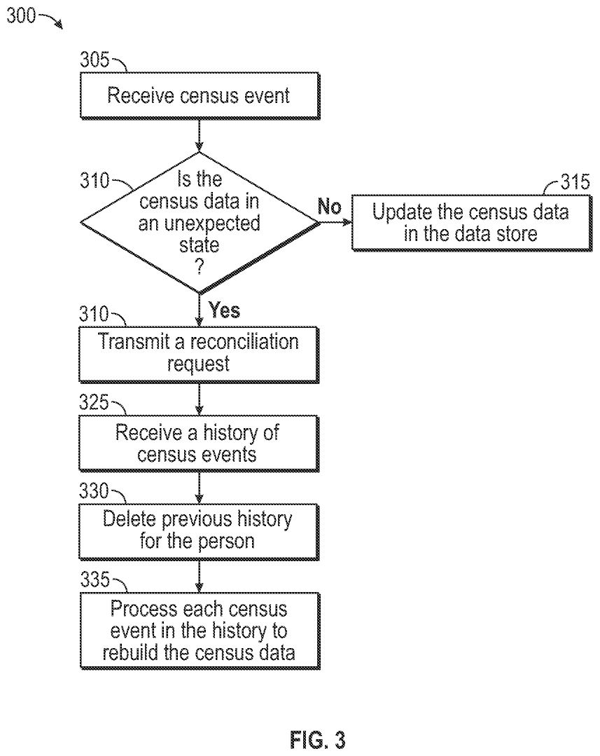

FIG. 3 is a flowchart of a method 300 for performing automatic reconciliation of census data between two computing systems, according to one embodiment. At block 305, a computing system (e.g., the second computing system 110 in FIGS. 1 and 2) receives a census event. In one embodiment, this census event corresponds to a change in a census state for a person (e.g., a patient or resident in a healthcare facility). As discussed above, the computing system may receive the census event from a source of truth of the census data (e.g., the first computing system 105 in FIG. 1). The computing system maintains its own copy of the census data, and to ensure its copy matches the census data maintained by the source of truth, the source of truth can send a census event each time the census state for a person changes. For example, the census event may be generated when a person is admitted into a facility, moved to different location within the facility, or discharged. The census event could also correspond to a patient being moved from a first facility (e.g., a hospital) to a second facility (e.g., a rehabilitation center).

In one embodiment, the computing system receives the census events from a message queue (e.g., the message queue 115 in FIG. 1). The message queue can forward the census events to the computing system, as well as any other computing systems that wish to maintain copies of the census data provided by the source of truth. As explained above, the message queue can help to ensure that the computing system is not overwhelmed by census updates. Further, the message queue can serve as a central repository for storing the census events and handle the task of distributing the census events to multiple destinations, thereby freeing computing resources at the source of truth.

At block 310, the error detector at the computing system (e.g., the error detector 215 in FIG. 2) determines whether the received census event causes the census data for the patient to be in an unexpected census state. The unexpected state can be any update that does not make sense in view of the other census events for the patient stored at the computing system. For example, the census event may indicate that a patient has been discharged from the hospital, but according to the census data maintained by the computing system, that patient has not yet been admitted into the hospital. Or the census event may indicate that the patient is in an operation recovery unit but according to the census data the patient has not yet been in an operation room.

In one embodiment, the error detector uses processing rules (e.g., the processing rules 220 in FIG. 2) to determine whether the received census event causes the patient to have an unexpected census state. Some census states may have respective processing rules that list one or more census events that should occur before the current census event. For example, the processing rules can list one, two, or three prerequisite census events that should have occurred before the currently received census event. If the processing rule lists multiple prerequisite census events, it can also indicate the order those events should occur. If the error detector determines that a prerequisite event did not occur, or the prerequisite events happened in the wrong order, the error detector can determine the received census event causes the patient to have an unexpected census state. Put differently, the census event indicates the patient is at an unexpected location, or has an unexpected status, relative to the census events stored locally at the computing system.

However, not all census events may have a corresponding processing rule. For example, census events indicating a patient was first admitted into a facility may not have any prerequisite census events, and thus, do not need a processing rule. Thus, these census events can be processed without using a processing rule since they cannot cause the patient to be in an unexpected census state.

In one embodiment, determining that the patient has an unexpected census state indicates there is a discrepancy between the census data stored locally at the computing system and the census data at the source of truth. For example, the computing system may have not received one of the census events for the patient, or the computing system may have received the census events in a different order than they actually occurred.

If the census event does not cause the patient to have an unexpected census state, the method 300 proceeds to block 315 where the computing system updates its local copy of the census data in the data store. That is, the census event may have satisfied the corresponding processing rule (e.g., the census data for the patient includes the prerequisite census events listed in the rule). In one embodiment, the error detector updates the EHR for the patient to include the census event.

However, if the census event causes the patient to have an unexpected census state, the method 300 proceeds to block 320 where a reconciliation engine (e.g., the reconciliation engine 225 in FIG. 2) transmits a reconciliation request to the source of truth of the census data. The reconciliation request can indicate the patient who has the unexpected census state due to the recently received census event. The reconciliation request can be sent through the message queue or directly to the source of truth via a network.

At block 325, the computing system receives a history of census events for the patient from the source of truth. In one embodiment, the source of truth transmits the EHR for the patient that may have a complete list of census events for the patient. In another embodiment, the source of truth may send a full list of census events, which may not include other medical information typically found in an EHR. In any case, the history of census events (or the EHR) can be sent along a path in the network that bypasses the message queue. That is, when performing reconciliation, the computing system can receive the history of census events from the source of truth without the history being received, or forwarded, by the message queue. This may reduce the time the computing system has to wait until it receives the history of census events.

In one embodiment, the reconciliation can be performed without the computing system receiving a full list of census events for the patient. In one embodiment, the source of truth may send a subset of the list of census events for the patient, e.g., only the census events that occurred in the last two days. The computing system can review the subset of census events and determine whether there is a discrepancy with its list of census event for that patient over the same two day time period. If so, the computing system can perform the blocks below to update its census data and determine whether the patient no longer has an unexpected census state. If not, the computing system can then send another reconciliation request to the source of truth, which in response can send the entire list of census events, or send a larger subset of the census events, e.g., the census events that occurred in the last week. Transmitting a subset of the census events may reduce the load on the network if the discrepancy can be resolved without having to send the entire list of census events—e.g., the full history of the patient.

At block 330, the computing system deletes a previous history for the person. If the computing system received a full history of the patient at block 325, then the computing system deletes the entire history of the patient (e.g., all the census events) stored locally in its data store. However, if the computing system received a subset of the census events from the source of truth (e.g., the census events within the last two days), the computing system deletes from its local data store any census events for the patient occurring over the same two day time period.

At block 335, a rebuilder in the computing system (e.g., the rebuilder 230 in FIG. 2) processes each census event in the history received from the source of truth to rebuild the census data stored in the computing system's data store. That is, after the previous history is deleted, the rebuilder can replace that history with the history received from the source of truth. As such, once the rebuilder has completed rebuilding the census data in the local data store, the census data for the patient at the local data store matches or mirrors the census data for the patient at the source of truth.

If the computing system received only a partial history from the source of truth (e.g., the census events for the last week), the error detector can reevaluate the rebuilt history to determine whether the patient still has an unexpected census state. If not, the automatic reconciliation process is complete. However, if the patient still has an unexpected census state, the reconciliation engine can request more census events from the source of truth which the rebuilder then uses to rebuild another portion of the patient's locally stored census data. In one embodiment, the computing system can continue to request more batches of census events from the source of truth until eventually the discrepancy is fixed and the person no longer has an unexpected census state.

In this manner, the method 300 describes an automatic reconciliation process that permits the computing system to recover from a discrepancy between its locally stored census data and the census data in the source of truth. The method 300 can advantageously transmit relatively lightweight census events to the computing system at block 305 to update the locally stored census data to match the census data at the source of truth. However, when a discrepancy is detected at block 310, the method can then automatically perform reconciliation where a history containing multiple census events is transmitted between the two computing systems in order to correct the discrepancy at blocks 320 and 325. In this manner, the strain on the network and the computing system is reduced relative to a system that transmits the full history of a patient each time their census state changes. Moreover, the method 300 can perform reconciliation without any human intervention which means discrepancies can be corrected almost as soon as they are detected and the history is received.

In one embodiment, the method 300 provides a prophylaxis treatment or a medical diagnosis for the patient by performing an automatic reconciliation process. As mentioned above, ensuring the census data at the computing system matches the census data at the source of truth helps the computing system to make more informed and better suggestions for the treatment of the patient. For instance, the computing system may be a marketing application that provides suggestions for follow-up care or different medical operations that can be used as part of a prophylaxis treatment or a medical diagnosis. For example, by knowing from the census data the patient has just completed an operation, the marketing application can generate a list of rehabilitation centers that specialize in the recovery of patients who underwent that same operation. In this manner, the census data can be used to provide a prophylaxis treatment or a medical diagnosis to the patient since the marketing application has access to up-to-date and accurate census data as a result of performing the method 300.

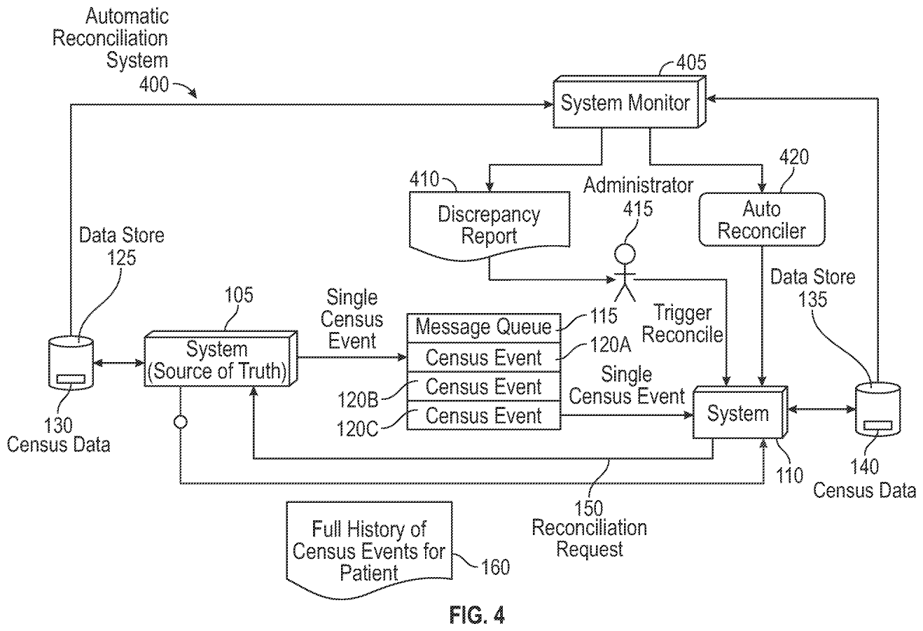

FIG. 4 illustrates a block diagram of an automatic reconciliation system 400, according to one embodiment. The system 400 includes many of the same components illustrated in FIG. 1 as indicated by using the same reference numbers. For brevity, these components are not described again here.

The system 400 also includes a system monitor 405 and an auto reconciler 420, which can both be software applications that execute on the same or different computing system. For example, the system monitor 405 and the auto reconciler 420 may execute on different (or the same) servers, cloud computing environments, or data centers.

As shown, the system monitor 405 is in communication with the data store 125 for the first computing system 105 and the data store 135 for the second computing system 110. The system monitor 405 can request or receive, at intervals, the census data 130, 140 stored in the data stores 125, 135. For example, every few hours or once a day, the system monitor 405 can retrieve the census data 130, 140 and compare the census data 130, 140 to identify discrepancies. In one embodiment, the system monitor 405 can, for each person, identify the census events stored in the census data 130 and determine whether those events match the census events stored in the census data 140. Further, the system monitor 405 can determine whether the census events are listed in the correct order.

Thus, the system monitor 405 can perform an additional check to ensure the census data 140 matches the census data 130. Unlike in the embodiments above where discrepancies were determined each time a new census event was received (i.e., in response to a change in a person's census state), the system monitor 405 can evaluate the census data 130, 140 at a predefined interval (e.g., every six hours or every night) to identify discrepancies. Further, while the method 300 determined whether there was a discrepancy in an individual person's census data, the system monitor 405 can perform a system-wide census check to look for discrepancies in every person's census data.

In one embodiment, the system monitor 405 generates a discrepancy report 410 listing the discrepancies found during the system-wide census check. The discrepancy report 410 can list a person (or patient) and describe the discrepancy between the census data 130, 140. For example, the report 410 could indicate that in census data 140, Patient A is missing a census event that is in census data 130. Or the report could indicate that in census data 140, Patient B has two census events that occurred in a different order in the census data 130. The census report 410 could also list all the census events from both the census data 130 and the census data 140 for the patients that have discrepancies in their histories. Doing so can provide context for a system administrator 415 tasked for resolving the discrepancies.

The system administrator 415 can decide which discrepancies to resolve and which discrepancies should be ignored. For example, the system administrator 415 may choose to resolve all the discrepancies by replacing the census events for the patients who had discrepancies in the census data 140 with the census events for those patients stored in the census data 130 (e.g., the source of truth). However, some discrepancies may be intentional and do not need to be reconciled. Thus, in this example, the system monitor 405 can facilitate a manual system-wide reconciliation by generating the discrepancy report 410.

Alternatively, the system 400 can perform an automatic system-wide reconciliation using the auto reconciler 420. In that case, the system monitor 405 transmits the discrepancies it identifies to the auto reconciler 420 which can then send reconciliation actions to the system 110. For example, the rebuilder (not shown) in the system 110 can then rebuild the census events for the patients who had a discrepancy in response to the actions transmitted by the auto reconciler 420. For example, the auto reconciler 420 can issue reconciliation actions for reconciling all the discrepancies identified by the system monitor 405. Thus, in this example, the system monitor 405 and the auto reconciler 420 can facilitate an automatic system-wide reconciliation by transmitting reconciliation actions to the rebuilder in the computing system 110.

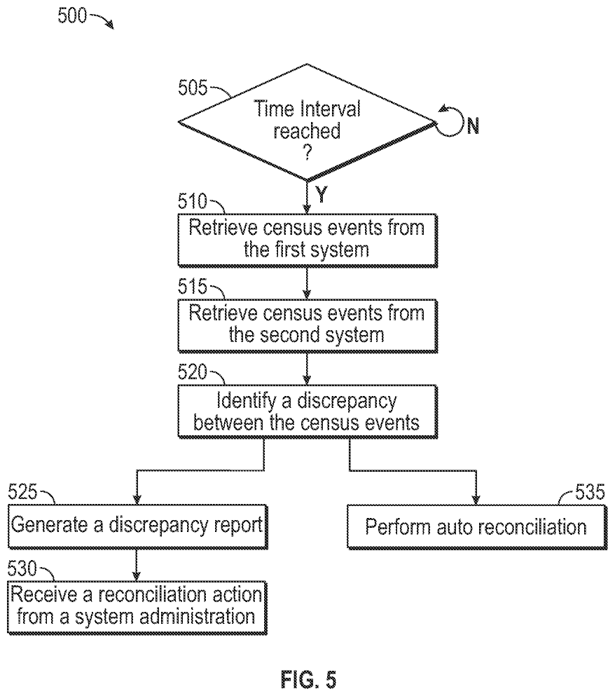

FIG. 5 is a flowchart of a method 500 for periodically comparing census states in two copies of census data to identify discrepancies, according to one embodiment. At block 505, the system monitor (e.g., the system monitor 405 in FIG. 4) determines whether a time interval for performing a system-wide check of the census data has been reached. That is, the system monitor may perform the method 500 at a predefined time interval such as once a day or once every two days. Further, the time interval may be set so that the check is performed during a time of day when there is traditionally low demand on the computing systems (e.g., late at night or on holidays). That way, performing the system-wide check has less of an impact on the normal operations of the computing systems.

At block 510, the system monitor retrieves census events from the first system—e.g., the census events of persons stored in the census data for the source of truth. In one embodiment, the system monitor retrieves census events from all the persons listed in the census data. However, in another embodiment, the system monitor may retrieve the census events for only some of the persons in the census data. For example, the system monitor may retrieve the census events for only the persons whose history has changed since the last time the system monitor performed a system-wide check. The system monitor may check each patient history in the census data to determine whether there is a census event that was added since the last time the system monitor performed a check, and if not, the system monitor does not retrieve the census data for that patient. That is, the system monitor may assume there are no discrepancies in the persons' history since those discrepancies would have been corrected in the previous system-wide check, and no new census events have been added in the meantime. Evaluating only the persons who have updated census data since the last time the system-wide check was performed may reduce the amount of computing resources required (or reduce the time required) to perform the check.

At block 515, the system monitor retrieves census events from the second system—e.g., the census events of persons stored the computing system that is intended to mirror the census data stored in the source of truth. In one embodiment, the system monitor retrieves the census events for the same persons whose census events were retrieved at block 510. For example, at block 510 if the system monitor retrieved the census events for only some of the persons in the census data, then at block 515 the system monitor retrieves census events for only those same persons. That way, the system monitor can compare the census events for the same persons stored at the two computing systems.

At block 520, the system monitor identifies a discrepancy between the census events. In one embodiment, for each person, the system monitor compares the corresponding census events stored in one data store (e.g., data store 125) with the corresponding census events stored at the other data store (e.g., data store 135). If the census events match—e.g., they include the same census events in the same order—the system monitor determines the census state for that person in both data stores matches. However, if one data store has a census event that is not in the other data store, or multiple census events occur in a different order, the system monitor determines there is a discrepancy in the census state for that person in the two data stores.

While the method 500 discusses comparing the census events stored in the data stores for each person, in another embodiment, the system monitor could just compare the census state of the person in both data stores to determine whether there is a discrepancy. That is, instead of comparing multiple census events for the person stored in both data stores, the system monitor could retrieve at blocks 510 and 515 only the current census state of the person from both data stores (e.g., the current location of the person or the current status of the person). If the census states for the person are the same in both the data stores, then the system monitors assumes the census events for the person also match. However, if the census states do not match, then the system monitors knows there is a discrepancy between the census events for that person in the two data stores (e.g., a census event is missing, or census events are out of order).

Once a discrepancy as identified, the method 500 can perform a manual reconciliation process as illustrated by blocks 525 and 530 or an automatic reconciliation process as illustrated in block 535.

At block 525, the system monitor generates a discrepancy report that is provided to a system administrator. As mentioned above, the report can list the discrepancies found during the system-wide check such as each person where a discrepancy was found and a description of the discrepancy. For example, the report could indicate that in the census data stored at the source of truth, Patient A has a census event that is not stored in the census data stored at the computing system. Or the report could indicate that in census data stored at the source of truth, Patient B has two census events that occurred in a different order than in the census data stored at the computing system.

The system monitor can then transmit this report to a system administrator for review. The system administrator can identify which discrepancies should be reconciled and which should not. The discrepancy report can provide context for a system administrator tasked for resolving the discrepancies, such as the number of discrepancies for each person, the types of discrepancies identified, and potential reconciliation actions that can be performed.

At block 530, the rebuilder in the computing system can receive a reconciliation action from the system administrator. The rebuilder can then, for example, perform the same actions as described at blocks 330 and 335 of the method 300 to rebuild the census data to complete the reconciliation process.

If instead the system monitor performs auto reconciliation, after block 520 the method 500 proceeds to block 535 where the auto reconciler (e.g., the auto reconciler 420) generates reconciliation actions that are transmitted to the rebuilder in the computing system. In one embodiment, the auto reconciler can determine using predefined rules which discrepancies identified by the system monitor should be reconciled and which should not. Alternatively, the auto reconciler can generate reconciliation actions to reconcile all the identified discrepancies. Like with the manual process, during auto reconciliation the rebuilder in the computing system can be leveraged to complete the reconciliation process.

### Example Computing Hardware

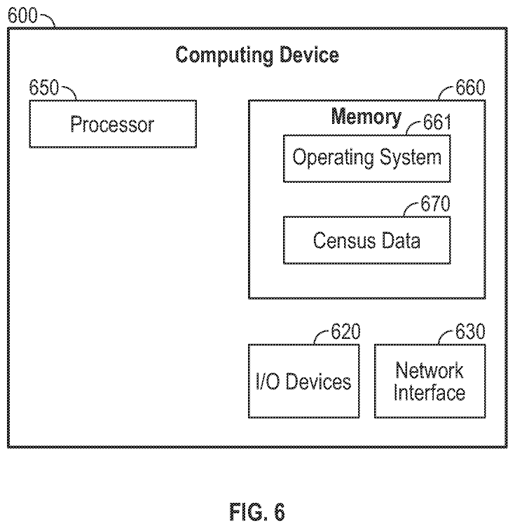

FIG. 6 illustrates a computing system 600, which may be used to implement various computing elements in the automatic reconciliation systems 100 and 400 in FIGS. 1 and 4 (e.g., a computer, a laptop, a tablet, a smartphone, web server, data center, cloud computing environment, etc.), or any other computing device described in the present disclosure. As shown, the computing system 600 includes, without limitation, a processor 650 (e.g., a central processing unit), a network interface 630, and memory 660. The computing system 600 may also include an I/O device interface connecting I/O devices 620 (e.g., keyboard, display and mouse devices) to the computing system 600.

The processor 650 retrieves and executes programming instructions stored in the memory 660 (e.g., a computer readable medium). Similarly, the processor 650 stores and retrieves application data residing in the memory 660. An interconnect facilitates transmission, such as of programming instructions and application data, between the processor 650, I/O device interface, storage, network interface 630, and memory 660. The processor 650 is included to be representative of a single processor, multiple processors, a single processor having multiple processing cores, and the like. And the memory 660 is generally included to be representative of volatile and non-volatile memory elements. For example, the memory 660 can include random access memory and a disk drive storage device. Although shown as a single unit, the memory 660 may be a combination of fixed and/or removable storage devices, such as magnetic disk drives, flash drives, removable memory cards or optical storage, network attached storage (NAS), or a storage area-network (SAN). The storage may include both local storage devices and remote storage devices accessible via the network interface 630. In this example, the memory 660 (e.g., storage) includes census data 670 (e.g., census data 130 or 140 in FIG. 1).

Further, the computing system 600 is included to be representative of a physical computing system as well as virtual machine instances hosted on a set of underlying physical computing systems. Further still, although shown as a single computing device, one of ordinary skill in the art will recognize that the components of the computing system 600 shown in FIG. 6 may be distributed across multiple computing systems connected by a data communications network.

As shown, the memory 660 includes an operating system 661. The operating system 661 may facilitate receiving input from and providing output to various components. For example, the network interface 630 can be used to communication between the computing systems 105 and 110, or to communicate between the system monitor 405 and the computing systems 105 and 110.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

The following clauses describe various embodiments of the present disclosure.

Clause 1: A method comprising receiving, from a source of truth via a network, a census event for updating census data stored at a computing system, the census data defining a census state for a person; determining that the census event causes the person to have an unexpected census state; transmitting, via the network, a reconciliation request to the source of truth; receiving, via the network, a first history of census events from the source of truth corresponding to the person; deleting a second history of census events stored at the computing system for the person; and processing the first history of census events to rebuild the census data for the person stored at the computing system.

Clause 2: In addition to the method of clause 1, the method further comprising receiving from the source of truth corresponding census events at the computing system every time the source of truth updates locally stored census data so that the census data at the computing system mirrors the locally stored census data at the source of truth.

Clause 3: In addition to the method of clause 2, the method further comprising receiving, at the computing system, the corresponding census events from a message queue communicatively coupled between the source of truth and the computing system.

Clause 4: In addition to the method of clause 3, wherein the first history of census events comprises all the census events for the person stored at the source of truth, wherein the first history of census events is transmitted to the computing system along a path in the network that bypasses the message queue.

Clause 5: In addition to the method of clause 4, wherein the first history of census events is part of an electronic health record (EHR) for the person that contains all the census events for the person, wherein the EHR is transmitted from the source of truth to the computing system in response to the reconciliation request.

Clause 6: In addition to the method of clauses 1, 2, 3, 4, or 5, wherein determining that the census event causes the person to have the unexpected census state comprises: identifying a processing rule corresponding to the census event, the processing rule listing at least one prerequisite census event; and upon determining that the prerequisite census event is not in the census data stored at the computing system for the person, determining that the census event causes the person to have the unexpected census state.

Clause 7: In addition to the method of clause 6, wherein the processing rule lists a plurality of prerequisite census events and a required order of the plurality of prerequisite census events that, if not met by the census data stored at the computing system, indicate the census events has caused the person to have the unexpected census state.

Clause 8: In addition to the method of clauses 1, 2, 3, 4, 5, 6, or 7, after processing the first history of census events to rebuild the census data for the person stored at the computing system, the method further comprising generating a suggestion for prophylaxis treatment for the person based on the census data or a medical diagnosis for the person based on the census data.

Clause 9: In addition to the method of clauses 1, 2, 3, 4, 5, 6, 7, or 8, the method further comprising, at intervals: retrieving census data for multiple persons from the source of truth; retrieving census data for the multiple persons from the computing system; identifying at least one discrepancy between the census data for a first person of the multiple persons in the source of truth and the census data for the first person in the computing system; and performing a reconciliation process to update the census data for the first person in the computing system to correct the discrepancy.

Clause 10: In addition to the method of clause 9, the method further comprising selecting the multiple persons by identifying the census data in the source of truth where a new census event has been added to a person's history since the last time a system-wide census check was performed.

Clause 11: A non-transitory computer readable medium comprising instructions to be executed in a processor, the instructions when executed in the processor perform an operation, the operation comprising: receiving, from a source of truth via a network, a census event for updating census data stored at a computing system, the census data defining a census state for a person; determining that the census event causes the person to have an unexpected census state; transmitting, via the network, a reconciliation request to the source of truth; receiving, via the network, a first history of census events from the source of truth corresponding to the person; deleting a second history of census events stored at the computing system for the person; and processing the first history of census events to rebuild the census data for the person stored at the computing system.

Clause 12: In addition to clause 11, the operation further comprises: receiving from the source of truth corresponding census events at the computing system every time the source of truth updates locally stored census data so that the census data at the computing system mirrors the locally stored census data at the source of truth.

Clause 13: In addition to clause 12, wherein receiving the corresponding census events from the source of truth further comprises: receiving, at the computing system, the corresponding census events from a message queue communicatively coupled between the source of truth and the computing system.

Clause 14: In addition to clauses 11, 12, or 13, wherein determining that the census event causes the person to have the unexpected census state comprises: identifying a processing rule corresponding to the census event, the processing rule listing at least one prerequisite census event; and upon determining that the prerequisite census event is not in the census data stored at the computing system for the person, determining that the census event causes the person to have the unexpected census state.

Clause 15: In addition to clause 14, wherein the operation further comprises, at intervals: retrieving census data for multiple persons from the source of truth; retrieving census data for the multiple persons from the computing system; identifying at least one discrepancy between the census data for a first person of the multiple persons in the source of truth and the census data for the first person in the computing system; and performing a reconciliation process to update the census data for the first person in the computing system to correct the discrepancy.

Clause 16: A system comprising: a processor; and memory storing code which, when executed by the processor, performs an operation, the operation comprising: receiving, from a source of truth via a network, a census event for updating census data stored at a computing system, the census data defining a census state for a person; determining that the census event causes the person to have an unexpected census state; transmitting, via the network, a reconciliation request to the source of truth; receiving, via the network, a first history of census events from the source of truth corresponding to the person; deleting a second history of census events stored at the computing system for the person; and processing the first history of census events to rebuild the census data for the person stored at the computing system.

Clause 17: In addition to clause 16, wherein the operation further comprises: receiving from the source of truth corresponding census events at the computing system every time the source of truth updates locally stored census data so that the census data at the computing system mirrors the locally stored census data at the source of truth.

Clause 18: In addition to clause 17, wherein receiving the corresponding census events from the source of truth further comprises: receiving, at the computing system, the corresponding census events from a message queue communicatively coupled between the source of truth and the computing system.

Clause 19: In addition to clauses 16, 17, or 18, wherein determining that the census event causes the person to have the unexpected census state comprises: identifying a processing rule corresponding to the census event, the processing rule listing at least one prerequisite census event; and upon determining that the prerequisite census event is not in the census data stored at the computing system for the person, determining that the census event causes the person to have the unexpected census state.

Clause 20: In addition to clauses 16, 17, 18, or 19, wherein the operation further comprises, at intervals: retrieving census data for multiple persons from the source of truth; retrieving census data for the multiple persons from the computing system; identifying at least one discrepancy between the census data for a first person of the multiple persons in the source of truth and the census data for the first person in the computing system; and performing a reconciliation process to update the census data for the first person in the computing system to correct the discrepancy.

What is claimed is:

1. A method, comprising:
receiving, from a source of truth via a network, a census event for updating census data stored at a computing system, the census data defining a census state for a person;
determining that the census event causes the person to have an unexpected census state;
transmitting, via the network, a reconciliation request to the source of truth;
receiving, via the network, a first history of census events from the source of truth corresponding to the person;
deleting a second history of census events stored at the computing system for the person; and
processing the first history of census events to rebuild the census data for the person stored at the computing system.

2. The method of claim 1, further comprising:
receiving from the source of truth corresponding census events at the computing system every time the source of truth updates locally stored census data so that the census data at the computing system mirrors the locally stored census data at the source of truth.

3. The method of claim 2, wherein receiving the corresponding census events from the source of truth further comprises:
receiving, at the computing system, the corresponding census events from a message queue communicatively coupled between the source of truth and the computing system.

4. The method of claim 3, wherein the first history of census events comprises all the census events for the person stored at the source of truth, wherein the first history of census events is transmitted to the computing system along a path in the network that bypasses the message queue.

5. The method of claim 4, wherein the first history of census events is part of an electronic health record (EHR) for the person that contains all the census events for the person, wherein the EHR is transmitted from the source of truth to the computing system in response to the reconciliation request.

6. The method of claim 1, wherein determining that the census event causes the person to have the unexpected census state comprises:
identifying a processing rule corresponding to the census event, the processing rule listing at least one prerequisite census event; and
upon determining that the prerequisite census event is not in the census data stored at the computing system for the person, determining that the census event causes the person to have the unexpected census state.

7. The method of claim 6, wherein the processing rule lists a plurality of prerequisite census events and a required order of the plurality of prerequisite census events that, if not met by the census data stored at the computing system, indicate the census events has caused the person to have the unexpected census state.

8. The method of claim 1, further comprising, after processing the first history of census events to rebuild the census data for the person stored at the computing system:
generating a suggestion for prophylaxis treatment for the person based on the census data or a medical diagnosis for the person based on the census data.

9. The method of claim 1, further comprising, at intervals:
retrieving census data for multiple persons from the source of truth;
retrieving census data for the multiple persons from the computing system;
identifying at least one discrepancy between the census data for a first person of the multiple persons in the source of truth and the census data for the first person in the computing system; and
performing a reconciliation process to update the census data for the first person in the computing system to correct the discrepancy.

10. The method of claim 9, further comprising:
selecting the multiple persons by identifying the census data in the source of truth where a new census event has been added to a person's history since the last time a system-wide census check was performed.

11. A non-transitory computer readable medium comprising instructions to be executed in a processor, the instructions when executed in the processor perform an operation, the operation comprising:
receiving, from a source of truth via a network, a census event for updating census data stored at a computing system, the census data defining a census state for a person;
determining that the census event causes the person to have an unexpected census state;
transmitting, via the network, a reconciliation request to the source of truth;
receiving, via the network, a first history of census events from the source of truth corresponding to the person;
deleting a second history of census events stored at the computing system for the person; and
processing the first history of census events to rebuild the census data for the person stored at the computing system.

12. The non-transitory computer readable medium of claim 11, wherein the operation further comprises:
receiving from the source of truth corresponding census events at the computing system every time the source of truth updates locally stored census data so that the census data at the computing system mirrors the locally stored census data at the source of truth.

13. The non-transitory computer readable medium of claim 12, wherein receiving the corresponding census events from the source of truth further comprises:
receiving, at the computing system, the corresponding census events from a message queue communicatively coupled between the source of truth and the computing system.

14. The non-transitory computer readable medium of claim 11, wherein determining that the census event causes the person to have the unexpected census state comprises:
identifying a processing rule corresponding to the census event, the processing rule listing at least one prerequisite census event; and
upon determining that the prerequisite census event is not in the census data stored at the computing system for the person, determining that the census event causes the person to have the unexpected census state.

15. The non-transitory computer readable medium of claim 14, wherein the operation further comprises, at intervals:
retrieving census data for multiple persons from the source of truth;
retrieving census data for the multiple persons from the computing system;
identifying at least one discrepancy between the census data for a first person of the multiple persons in the source of truth and the census data for the first person in the computing system; and
performing a reconciliation process to update the census data for the first person in the computing system to correct the discrepancy.

16. A system, comprising:
a processor; and
memory storing code which, when executed by the processor, performs an operation, the operation comprising:
receiving, from a source of truth via a network, a census event for updating census data stored at a computing system, the census data defining a census state for a person;
determining that the census event causes the person to have an unexpected census state;
transmitting, via the network, a reconciliation request to the source of truth;
receiving, via the network, a first history of census events from the source of truth corresponding to the person;
deleting a second history of census events stored at the computing system for the person; and
processing the first history of census events to rebuild the census data for the person stored at the computing system.

17. The system of claim 16, wherein the operation further comprises:
receiving from the source of truth corresponding census events at the computing system every time the source of truth updates locally stored census data so that the census data at the computing system mirrors the locally stored census data at the source of truth.

18. The system of claim 17, wherein receiving the corresponding census events from the source of truth further comprises:
receiving, at the computing system, the corresponding census events from a message queue communicatively coupled between the source of truth and the computing system.

19. The system of claim 16, wherein determining that the census event causes the person to have the unexpected census state comprises:
identifying a processing rule corresponding to the census event, the processing rule listing at least one prerequisite census event; and
upon determining that the prerequisite census event is not in the census data stored at the computing system for the person, determining that the census event causes the person to have the unexpected census state.

20. The system of claim 16, wherein the operation further comprises, at intervals:
retrieving census data for multiple persons from the source of truth;
retrieving census data for the multiple persons from the computing system;
identifying at least one discrepancy between the census data for a first person of the multiple persons in the source of truth and the census data for the first person in the computing system; and
performing a reconciliation process to update the census data for the first person in the computing system to correct the discrepancy.
